(19) United States
(12) Patent Application Publication
(10) Pub. No.: US 2010/0280408 A1
(43) Pub. Date: Nov. 4, 2010

(54) FINE NEEDLE BIOPSY SYSTEM AND METHOD OF USE

(76) Inventor: Joseph G. Rusnak, East Aurora, NY (US)

Correspondence Address: Jaekle Fleischmann & Mugel, LLP, 12 Fountain Plaza, Buffalo, NY, 14202-2292, US

(21) Appl. No.: 12/432,792

(22) Filed: Apr. 30, 2009

(51) Int. Cl. A61B 10/02

(52) U.S. Cl. 600/567

(57) ABSTRACT

The present invention includes a biopsy needle system comprising a tubular cannula having a first longitudinal passage, a first length, and a first grip on the proximal end of the cannula. The tubular biopsy needle has a second longitudinal passage, a second length greater than the first length, an inward beveled distal end, and a second grip on the proximal end. The biopsy needle is both sized and configured to fit within the first passage.

### FIELD OF THE INVENTION

The present invention relates generally to tissue biopsy needles and the use to harvest tissue in a minimally invasive manner.

### BACKGROUND OF THE RELATED ART

The development of non-invasive assays for diagnosing certain types of cancer has provided practitioners with some options for diagnosis of cancer and other related disease without the need for tissue biopsy. However, such options are limited in their applications and effectiveness. Still, the most definitive test for cancer or related disease is tissue biopsy. Tissue biopsy occurs when a sample of tissue from a site that is believed to be diseased is harvested and analyzed by a pathologist to determine the nature of the tissue, whether it is healthy tissue, benign tissue growth, or cancerous tissue.

Tissue samples from small organs or glands like lymph nodes or thyroid glands are especially difficult to harvest due to the relatively small size of the organs or glands (and their respective growths or nodules), location in sites that are uncovered by everyday clothing, and proximity to significant blood vessels. Furthermore, vascularization in many glands or organs is considerable making the possibility of bleeding a significant issue.

The process for harvesting a tissue sample from a patient includes three types of procedure. First, open surgery occurs when a patient is cut open to expose the tissue from which a sample is required. The procedure is invasive and creates a considerable risk of infection and side effects as compared to less invasive means. Of the three procedures for biopsy, it is generally considered the least desirable.

The second procedure is use of a core tissue biopsy needle. A core tissue biopsy needle requires removal of a relatively large piece of tissue from the desired tissue site. The tissue sample is large enough that it requires further processing, including slicing of the tissue sample to analyze under a microscope.

An example of core biopsy device is U.S. Pat. No. 4,177,797. The apparatus comprises an improved rotary biopsy device for extracting biopsy samples and the like from specific specimen extracting sites. The biopsy device comprises a tubular member with a distal end, defining a cutting edge. The distal end has a bevel on the inside of the tubular member. The '797 patent indicated, in 1977, that the problem in the biopsy field was that prior biopsy devices had not been successful in providing high quality extracted specimens while at the same time minimizing trauma to the patient and providing easy removability of the specimen from the patient.

U.S. Pat. No. 5,005,585 discloses a core biopsy needle construction that has an elongated needle member with a sharp annular interior edge. The inner surface of the cannula, adjacent the distal end, is undercut to provide a sharp angular interior edge. A stylus is disposed within the cannula and has an angular distal end. At the time of its filing, the '585 patent commented on the state of the art as follows, “In the past, biopsy procedures have been carried out using a hollow needle of varying internal diameter with a tapered distal end and a diagonal cutting edge . . . . Although biopsy needles of this construction have met with success, failure to obtain an adequate core biopsy specimen frequently occurs. Not infrequently, biopsy specimens will be left behind . . . thus necessitating the need for attempting another pass at obtaining a core biopsy.”

Another technique for harvesting tissue is fine needle aspiration (FNA). Fine needle aspiration removes smaller samples of biopsy tissue that can typically be viewed under the microscope without tissue slicing. The FNA procedure is as follows. A patient is given a local anesthetic. A needle (typically a 20-30 gauge needle) is inserted hypodermically and positioned to the site of the targeted tissue. The needle often penetrates fat tissue and muscular tissue depending upon the location of the targeted tissue. The organ or gland is held stationary relative to the movement of the needle. Then, the needle is gently moved into the tissue, thereby causing the needle to take a thin sample of the tissue. The tissue is drawn into the needle either by aspiration or by the capillary action of the needle. Aspiration occurs with the use of a vacuum source such as a syringe. To aid in harvesting an adequate sample size, the needle is optionally moved in and out of puncture site from about one to five times. This reciprocating motion causes cellular material to be scraped from the tissue and drawn into the needle.

Then, the needle is withdrawn from the patient and the tissue collected in the needle is placed on a slide for pathological analysis. Since the individual tissue specimens are smaller/thinner than a core biopsy, no additional tissue slicing is required. The sample can be placed on a slide, stained and analyzed. Often, with current FNA needles, the process is repeated to obtain between eight and eighteen slides with tissue samples.

U.S. Pat. No. 6,908,440 discloses a fine needle aspiration system with a first sharp edge at the beveled distal end of the needle that scrapes tissue during proximal to distal travel of the needle. The needle further comprises a second sharp edge defined by a transversely disposed slot formed in the needle near the distal end. The second sharp edge may be coincident, recessed, or elevated with the exterior surface of the needle. The '440 patent discusses a drawback of fine needle aspiration, “[T]he FNA [fine needle aspiration] biopsy needle procedure fails to collect a sample of sufficient size to enable definitive pathological results. When this happens, the physician must repeat the procedure causing additional trauma to the body part undergoing biopsy and creating additional risk of an adverse event.”

Kim, et al., “US-guided Fine-Needle Aspiration of Thyroid Nodules: Indications, Techniques, Results,” RadioGraphics, Volume 28, Number 7, pp. 1869-1899 (November 2008), is a review of current FNA results using ultrasound (US) versus palpation techniques. Despite many proposed designs for biopsy needles, standard hypodermic needles having a size of between 20 and 30 gauge are still the industry standard. Kim concludes, “However, the achievement of optimal results of the thyroid FNA, with increased efficacy and decreased inadequacy results, requires not only a skillful aspiration technique and attention to the factors that affect material adequacy but also awareness of the indications for and limitations of FNA biopsy . . . . US-guided FNA yields an inadequate specimen in 10%-20% of procedures . . . .”

After many years of attempting to develop a biopsy needle, that is an improvement over the hypodermic needle in efficiently obtaining adequate FNA quality samples and that minimize variation in biopsy procedure success caused by the level of practitioner experience, the present apparatus and method were developed to address these and other concerns.

### SUMMARY OF THE INVENTION

The present invention includes a biopsy needle system. The system comprises a cannula that has a first lumen, a first length, and a first grip on the proximal end of the cannula. The cannula is both sized and configured to receive a needle.

The system further includes a needle having a second lumen, a second length greater than the first length, an inner bevel on the distal end, and a second grip on the proximal end. The cannula has a sharpened or pointed distal end such as one found on a hypodermic needle or other cannula (known in the art) that are designed to enter tissue with as little trauma to the tissue as possible. The use of the cannula allows deployment through skin and even muscular tissue with minimal bleeding and bruising. Once the cannula is positioned at the tissue sampling site, the needle is deployed through the lumen of the cannula. The inner bevel defines a mouth that more efficiently receives tissue into the needle lumen by aspiration or by capillary forces.

In one embodiment, the first and second grip is a standard connector for a hypodermic needle such as a luer fitting.

In one embodiment, the system further comprises a trochar that is both sized and configured to fit in the cannula lumen. In another embodiment, the biopsy needle system has a cannula that is a minimum of 3 cm in length and a maximum of 15 cm in length.

In one embodiment, the biopsy needle system has a distal end of the biopsy needle that extends beyond the distal end of the cannula a minimum of 0.1 cm, 0.2 cm, or 0.4 cm and a maximum of about 3 cm, 1.8 cm, 1.6 cm or 1.0 cm.

The distal end of the biopsy needle of another embodiment has a cut away portion forming a longitudinally extending cutting edge that is configured to cut tissue in contact with the cutting edge upon rotation of the biopsy needle.

The cannula is an 16 gauge needle or smaller according to one embodiment and the biopsy needle is a 17 gauge needle or smaller according to another embodiment.

The present invention includes a method of sampling tissue that includes a biopsy needle system according to one or more of the embodiments disclosed herein. The cannula is inserted into the patient and positioned adjacent the site of tissue sampling. The biopsy needle is then inserted into the cannula. The needle is pushed into the tissue sampling site to receive tissue into the second passage of the biopsy needle. The deployment of the cannula minimizes tissue trauma during insertion. The tissue sample is removed from the needle. Optionally, another sample can be obtained by the same needle or a new needle by inserting the same needle or the new needle into the held stationary cannula. Then, when sampling is completed, the needle and cannula are removed from the patient.

In one embodiment, the distal end of the biopsy needle extends beyond the distal end of the cannula a minimum of 0.1 cm and a maximum of about 1 cm. The design prevents the needle from penetrating into the tissue sampling site a distance greater than the distance the needle extends beyond the cannula.

The distal end of the biopsy needle is further provided with a cut away portion forming a longitudinally extending cutting edge that is configured to cut tissue in contact with the cutting edge upon rotation of the biopsy needle. This method further comprises inserting the biopsy needle into the site to position the cutting edge against the tissue and rotating the biopsy needle.

In another embodiment, the tissue sampling site is on a thyroid gland.

In another embodiment, the biopsy needle system includes a trochar. The trochar is sized and configured to cooperate with the cannula to penetrate tissue with minimal damage. In one embodiment, the trochar and cannula form a conically shaped pointed tip.

Optionally, the method of operation of the system of one or more embodiment further includes inserting a trochar into the cannula, inserting the cannula and trochar into the patient, and removing the trochar from the cannula. Thereafter, in one embodiment, the biopsy needle is inserted into the cannula. The tip of the biopsy needle is positioned adjacent the tissue sampling site. The tissue is then harvested according to one or more examples and embodiments disclosed herein. Then, the needle and the cannula are removed from the patient.

In another embodiment, there is a kit with instructions: a tubular cannula that has a first longitudinal passage, a first length, and a first grip on the proximal end of the cannula, wherein the cannula is both sized and configured to receive a biopsy needle. There is also a tubular biopsy needle that has a second longitudinal passage, a second length greater than the first length, a beveled distal end, and a second grip on the proximal end. In one embodiment, there is instructions that direct a person to (1) insert the cannula into the patient and position the cannula adjacent the site of tissue sampling, (2) insert the biopsy needle into the cannula, (3) position the tip of the biopsy needle adjacent the site, (4) push the needle into the site of tissue sampling, (5) remove the needle, (6) remove the sample from the needle, (7) reinsert the needle or a new needle into the held stationary cannula, if needed, to obtain at least one additional sample, and (8) remove the cannula. Optionally, the kit further comprising a trochar that is both sized and configured to fit in the first passage.

### BRIEF DESCRIPTION OF THE DRAWINGS

FIG. 1 is a fine needle biopsy system of one embodiment of the invention.

FIG. 2 is an enlarged sectional view of the fine needle biopsy system of FIG. 1 taken along the lines of 2-2.

FIG. 3 is an end view of the biopsy system of FIG. 1.

FIG. 4 is the tip of a fine needle biopsy system of another embodiment of the invention.

FIG. 5 is a sectional view of the biopsy needle system of FIG. 4.

FIG. 6 is a sectional view of a cannula of FIG. 4 and the trochar used in one embodiment.

FIG. 7 is a sectional view of a cannula of FIG. 1 and the trochar used in one embodiment.

FIGS. 8-11 illustrate various steps of the use of the system of one or more embodiments of the present invention.

FIG. 12 is an illustration of the tip design of a biopsy needle of one embodiment.

### DETAILED DESCRIPTION

As noted, the present invention is a fine needle biopsy system that has improved delivery to the site of tissue biopsy. The biopsy system uses a cannula to deliver the needle to the tissue biopsy site. The needle is designed to more efficiently harvest or sample tissue. The device is capable of being positioned with minimal pain, tissue trauma, and bleeding. The harvesting of the tissue is capable of adequate sample size without unnecessary tissue damage.

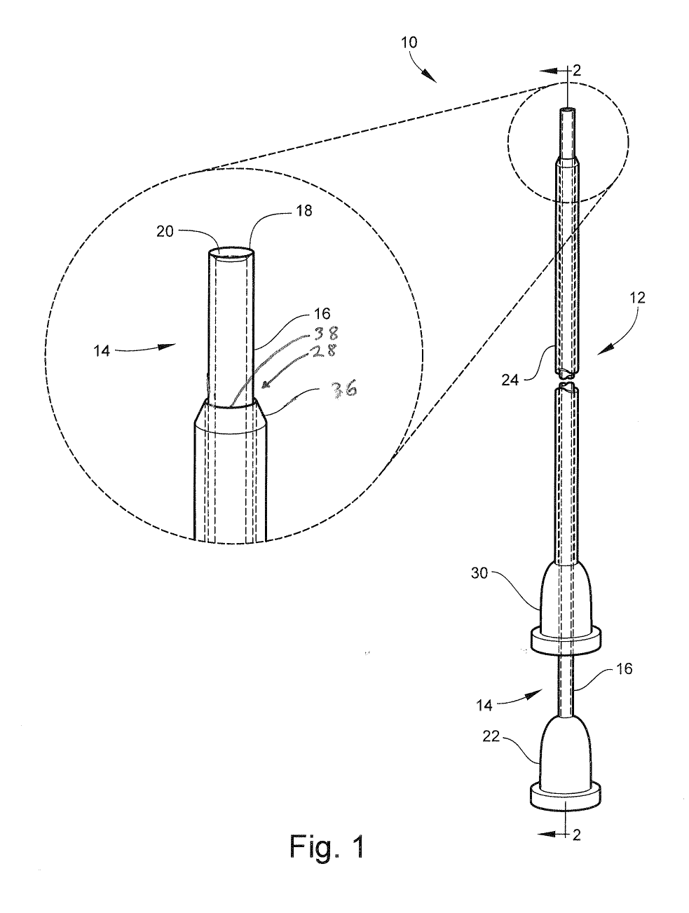

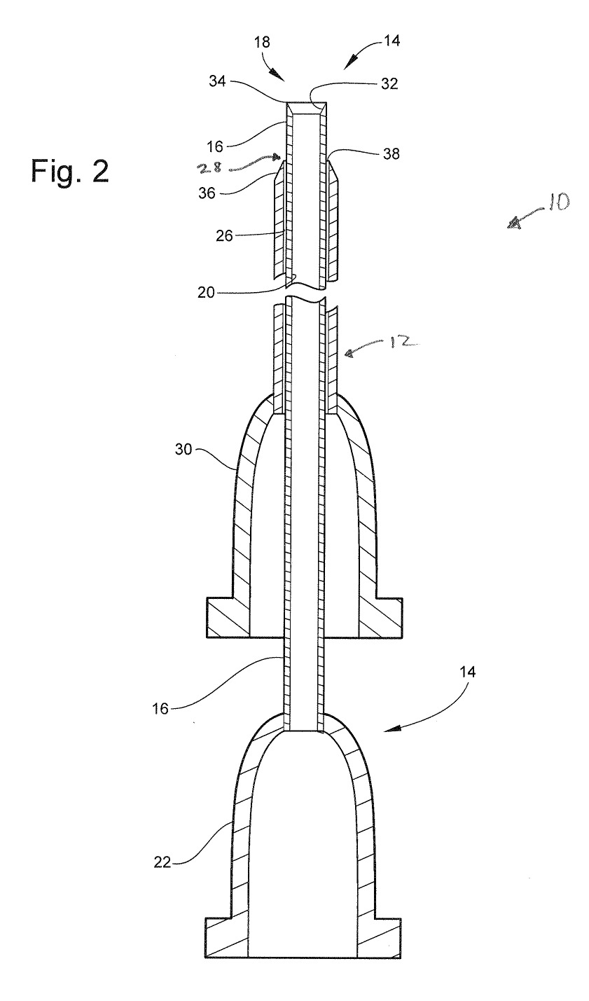

With reference to FIG. 1, the fine needle biopsy system 10 includes a cannula 12 and a needle 14. Also shown are a cutaway view in FIG. 2 and an enlarged distal end view in FIG. 3. The cannula 12 of one embodiment has a grip that comprises a luer fitting 30 and a tubular cannula shaft 24 that defines the cannula lumen 26.

The cannula tip 28 has an outer bevel 36 that causes the tip 28 of the cannula 12 to form a cutting edge 38 on the inside surface of the cannula 12 which forms a generally frusto-conical shape. An “outer bevel” as it pertains to a tubular member is defined as a bevel along the outer surface or edge of the end of the tubular member.

The needle 14 is both sized and configured to be received within the cannula lumen 26 from the proximal end where the luer fitting 30 (or other handle) is located to the distal end where the cannula tip 28 is located.

The needle 14 has a needle shaft 16 and a needle lumen 20 and a needle grip 22. In one embodiment, the needle grip 22 is a luer fitting. The needle 14 also has a needle tip 18.

With continued reference to FIGS. 1-3, the needle tip 18 has an inner bevel 32. An “inner bevel,” as it relates to a tubular object, is defined as a bevel of the inner surface of the end of a tubular object. The inner bevel 32 defines the cutting edge 34 of the needle 14 that is radially aligned with the outer surface of the needle shaft 16.

The inner bevel 32 of the needle 14 defines a cutting edge 34 that shaves or cuts tissue as it is moved in a forward direction through tissue, or alternatively along the surface of tissue, and a mouth into which sample tissue can be received in the needle lumen 20. More specifically, the cutting edge 34 of the of the needle 14 more effectively provides contact with the side of a puncture wound created by the entry of the cannula 12, the trochar (not shown in FIG. 1), or the needle 14 into the tissue harvest site. Forward motion of the needle 14 along the puncture wound will likewise more effectively shave tissue from the sides of the puncture wound to result in an aspirated sample which contains a sufficient amount of tissue to perform an effective biopsy.

The length of the shaft 16 of the needle 14 is longer than the cannula such that when the needle is inserted as far as possible into the shaft of the cannula, the needle tip 18 extends beyond the tip 28 of the cannula 12. In one embodiment, the needle extends a distance that is a minimum of 0.1 cm, 0.2 cm, or 0.4 cm and a maximum of 3 cm, 1 cm, 0.8 cm, or 0.7 cm. The relative length of the needle and the cannula ensure that the needle does not penetrate too far beyond the cannula.

With continued reference to FIGS. 1-3 and reference to FIG. 7, there is illustrated a trochar that is used in one embodiment with the cannula and needle of FIGS. 1-3. Illustrated in FIG. 7 is the cannula 12 having the cannula shaft 24 and the cannula tip 28. The cannula tip 28 has an outer bevel defining a frusto-conical shape that has the cutting edge 38 that is aligned with the inner surface of the cannula 12. The trochar 39 having a trochar shaft 40 and a trochar tip 42 is received within the cannula lumen 26. The tip 42 of the trochar 39 is conical. In one embodiment, the trochar tip is aligned with the tip of the cannula to define a conical piercing tip. The trochar proximal end fits into the first grip (not shown). In operation, the cannula 12 and the trochar 39 can be inserted into the patient with minimal tissue damage to the site of the tissue harvest. Optionally, the trochar may be used to penetrate into the sample tissue site to create a passage along which the tissue can be later removed by forward movement of the biopsy needle along the passage. Alternatively, the trochar and cannula are delivered to the site of the tissue sample, but do not penetrate into the tissue sample site.

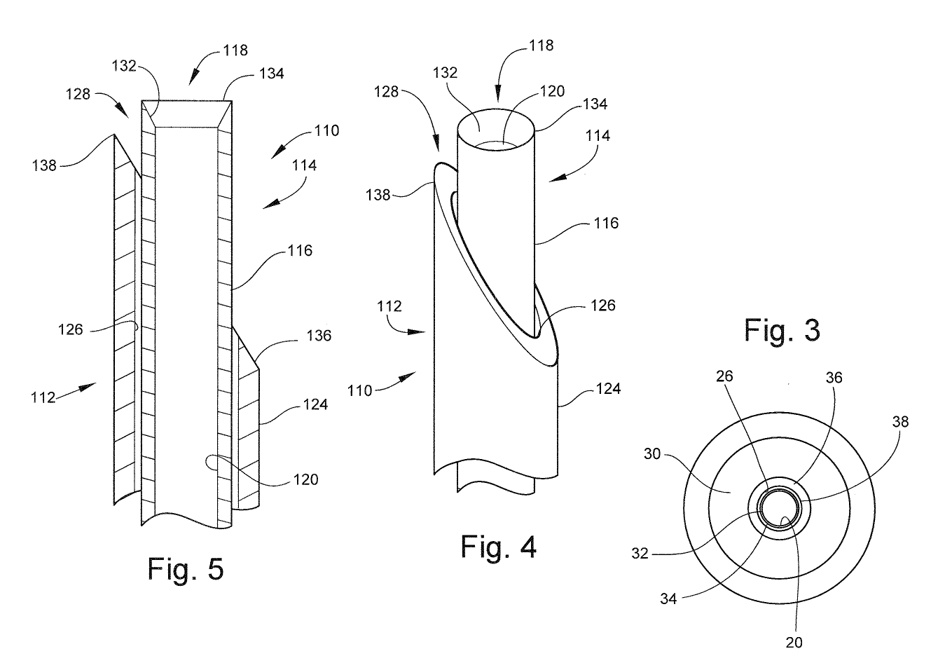

With reference to FIG. 4 and the corresponding cut away view of FIG. 5, a second embodiment of the fine needle biopsy system 110 includes a cannula 112 and a needle 114. The cannula 112 of one embodiment has a grip that comprises a luer fitting (not shown), a tubular cannula shaft 124, and a cannula lumen 126.

The cannula tip 128 has a slanted beveled surface defining a cutting edge 138 on one side of the cannula. By “slanted beveled surface” it is meant a bevel that extends on a slanted angle from one side of the tubular cannula 112 to the opposite side.

The needle 114 is both sized and configured to be received within the cannula lumen 126 from the proximal end where a luer fitting (not shown) or other handle is located to the distal end where the cannula tip 128 is located.

The needle 114 has a needle shaft 116 and a needle lumen 120 and a grip (not shown). In one embodiment, the needle grip 122 is a luer fitting. The needle 114 also has a needle tip 118.

The needle tip 118 has an inner bevel 132 with the cutting edge 134 that is radially aligned with the outer surface of the needle shaft 116.

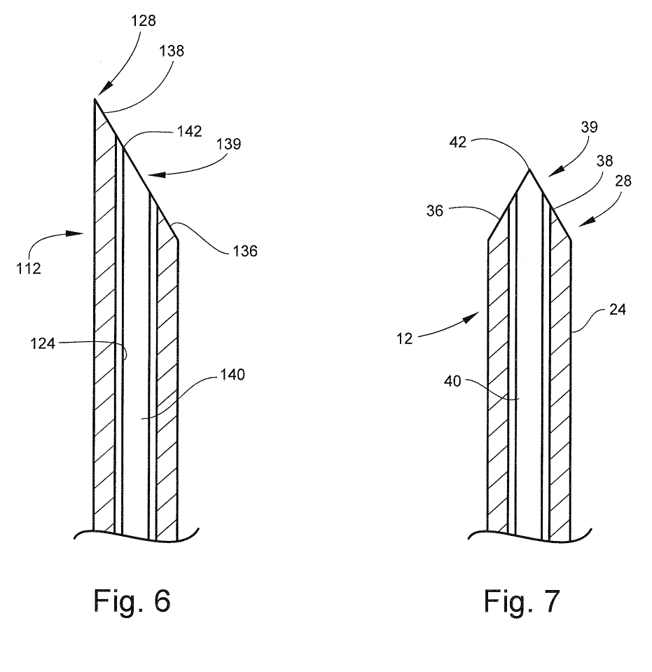

The inner bevel 132 of the needle 114 defines the cutting edge 134 that shaves or cuts tissue as it is moved in a forward direction through tissue, or alternatively along the surface of tissue, and a mouth into which sample tissue can be received in the needle lumen 120. With continued reference to FIGS. 4-5 and reference to FIG. 6, there is illustrated a trochar 39 that is used in one embodiment with the cannula and needle of FIGS. 4-5. Illustrated in FIG. 6 is a cannula 112 having a cannula shaft 124 and a cannula tip 128. The tip 128 of the cannula 112 has a side bevel.

A trochar 139 has a trochar shaft 140 and a trochar tip 142 that is received within the lumen 126 of the cannula. The tip 142 of the trochar 139 also has a side bevel of the same angle as the side bevel of the cannula 112. In one embodiment, the trochar tip 142 is aligned with the tip of the cannula 112. In operation, the cannula 112 and trochar 139 can be inserted into the patient with minimal tissue damage to the site of the tissue harvest. Optionally, the trochar 139 may be used to penetrate into the sample tissue site to create a passage along which the tissue can be later removed by forward movement of the biopsy needle along the passage. Alternatively, the trochar 139 and the cannula 112 are delivered to the site of the tissue sample, but do not penetrate into the tissue sample site.

An alternative tip design of the needle is illustrated with reference to FIG. 12. A needle 300 has a needle shaft 302 and a lumen 304. An inner bevel 305 is formed on the distal tip of the needle forming a cutting edge 306. Optionally, a longitudinally extending cut away portion defining an opening 307 defines a pair of longitudinally extended cutting edges 308 and 310. The cutting edges operate upon rotation when the needle 300 is inserted into the tissue sample site. The rotation of the needle shaft 302 causes the cutting edges 308 and 310 to contact the adjacent tissue, causing it to cut or shave the tissue into the cut away portion, where it can be drawn into the lumen 304 of the needle by capillary forces or by aspiration.

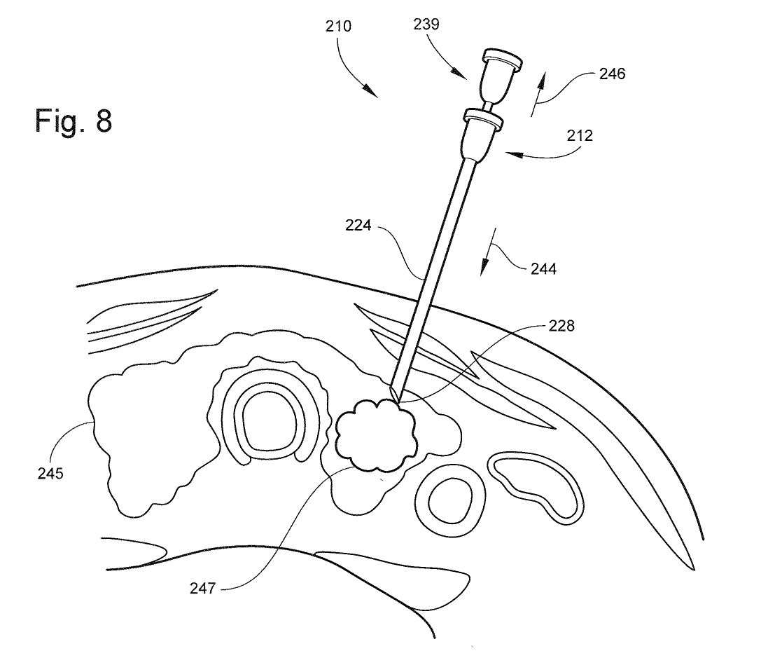

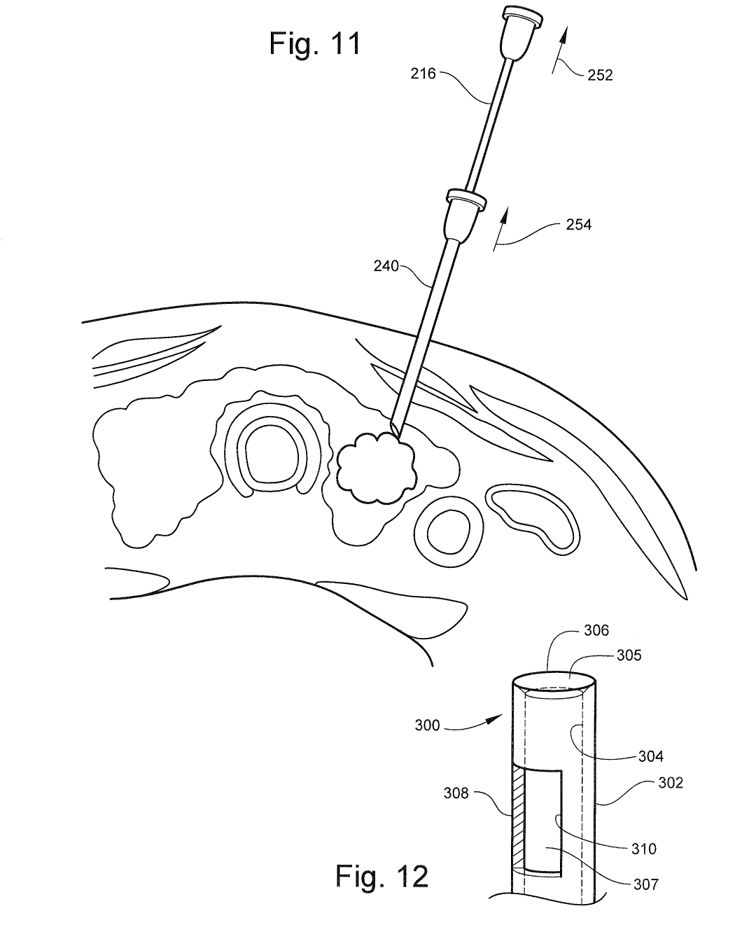

With reference to FIGS. 8-11, the methods of deployment and use are illustrated. Although the illustration involves a tissue biopsy on a thyroid nodule, it is understood that any desired tissue sample site could be targeted by the present invention.

FIG. 8 shows the first step in the deployment of the tissue biopsy system 210 including a cannula 212 and a trochar 239 within the lumen (not shown) of the cannula by penetrating the patient hypodermically in the direction of arrow 244. The cannula 224 and the trochar 239 together form a slanted tip 228, defining a cutting edge that allows deployment with minimal tissue damage and as small an entry wound as possible. Once the cannula 224 is positioned to the site of the tissue harvesting, the trochar 239 is removed in the direction of arrow 246. In one embodiment, the trochar 239 and the cannula 224 are pushed into the tissue harvest site and withdrawn to create a passage into which the needle (not shown) later penetrates.

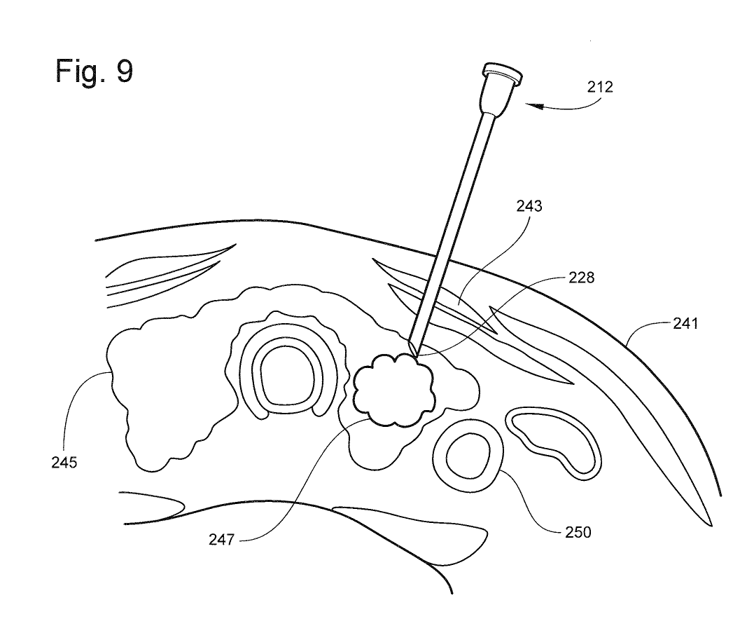

With reference to FIG. 9, the cannula 212 is in the deployed position showing that it passes through the patient's epidermis 241, muscular tissue 243, and tissue of the thyroid gland 245 either adjacent to or within the nodule 247. Avoidance of major blood vessels such as the carotid artery 250 is an important part of successful deployment of the system of the present invention. In one embodiment, ultrasound visualization is used to assist in the safe deployment of the cannula 212.

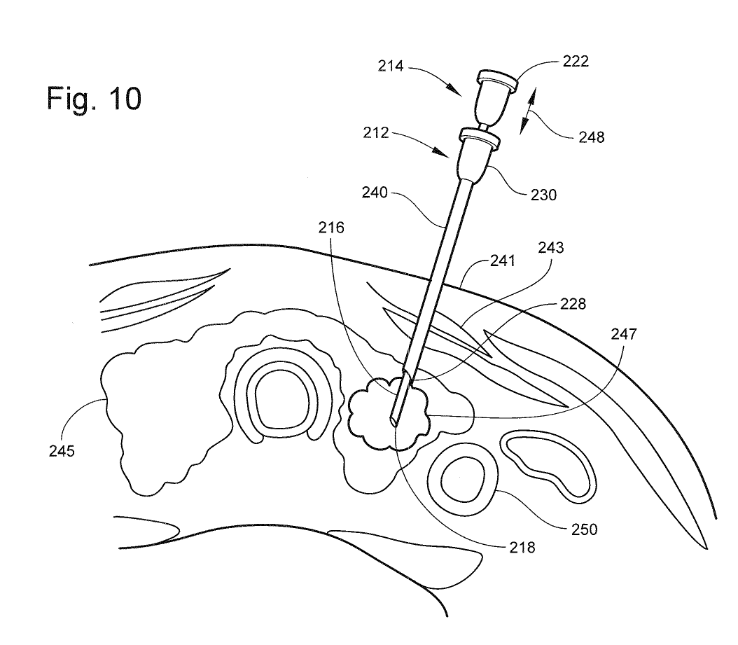

With reference to FIG. 10, a needle 214 of one or more embodiments of the present invention is inserted into the lumen (not shown) of the cannula 212. The operator grips the luer fitting 222 of the needle 214 and the luer fitting of the cannula and then pushes the needle into the cannula. As the needle tip 218 is pushed past the tip 228 of the cannula 212, tissue harvesting begins as the cutting edge cuts or shaves tissue into the lumen (not shown) of the needle. The tissue can be drawn in through aspiration or capillary forces of the needle 214. Although not shown, it is well within the scope of the present invention to have a hypodermic syringe affixed to the luer fitting of the needle. As needed, the needle can be repeatedly inserted and withdrawn into the tissue site as illustrated by direction arrow 248. In one embodiment, such as, for example, the device of FIG. 10, the tissue is harvested by a forward motion of the needle 214 into the tissue sampling site. In another embodiment, such as for example the device illustrated in FIG. 12, tissue is harvested by placing the opening 307 in the needle shaft 302 adjacent the tissue sampling site and rotating the needle 300.

After tissue harvesting is completed, the needle shaft 216 is withdrawn in the direction of arrow 252. The cannula shaft 240 is likewise withdrawn in the direction of the shaft 254 either simultaneously or sequentially with the needle shaft 216.

In another embodiment, the present invention further includes a kit that has a tubular cannula having a first longitudinal passage, a first length, and a first grip on the proximal end of the cannula, wherein the cannula is both sized and configured to receive a biopsy needle. The kit further includes a tubular biopsy needle having a second longitudinal passage, a second length greater than the first length, a beveled distal end, and a second grip on the proximal end. The kit contains instructions that direct a person to (1) insert the cannula into the patient and position the cannula adjacent the site of tissue sampling, (2) insert the biopsy needle into the cannula, (3) position the tip of the biopsy needle adjacent the site, (4) push the needle into the site of tissue sampling to obtain a sample in the needle, (5) perform further aspiration biopsy(s) using a new biopsy needle or the same biopsy needle after the tissue sample has been removed by inserting the biopsy needle into the cannula which has been held stationary, and (6) remove the cannula. Optionally, the kit further comprises a trochar that is both sized and configured to fit in the passage.

While the foregoing illustrations, examples, and description are intended to teach a person of ordinary skill, variations and combinations well within the scope of a skilled artisan are intended to be encompassed in the present invention. The foregoing disclosure is in no way intended to limit the scope and meaning of the invention except as defined by the claims which follow hereafter.

What is claimed is:

1. A biopsy needle system comprising:
a tubular cannula having a first longitudinal passage, a first length, and a first grip on the proximal end of the cannula; and
a tubular biopsy needle having a second longitudinal passage, a second length greater than the first length, an inward beveled distal end, and a second grip on the proximal end, wherein the biopsy needle is both sized and configured to fit within the first passage.

2. The biopsy needle system of claim 1, further comprising a trochar that is sized and configured to fit in the passage.

3. The biopsy needle system of claim 1, wherein the cannula is a minimum of 3 cm in length and a maximum of 15 cm in length.

4. The biopsy needle system of claim 1, wherein the distal end of the biopsy needle extends beyond the distal end of the cannula a minimum of 0.1 cm and a maximum of about 3 cm.

5. The biopsy needle system of claim 1, wherein the distal end of the biopsy needle has a cut away portion forming a longitudinally extending cutting edge that is configured to cut tissue in contact with the cutting edge upon rotation of the biopsy needle.

6. The biopsy needle system of claim 1, wherein the cannula is an 16 gauge needle or smaller.

7. The biopsy needle system of claim 1, wherein the biopsy needle is a 17 gauge needle or smaller.

8. A method of sampling tissue comprising:
providing a biopsy needle system of claim 1;
inserting the cannula into the patient and positioning the cannula adjacent the site of tissue sampling;
inserting the biopsy needle into the cannula;
positioning the tip of the biopsy needle adjacent the site;
moving the needle in a reciprocating motion within the cannula, wherein the reciprocating motion causes the distal end of the needle to cut tissue in the site and receive the tissue within the second passage; and
removing the needle and cannula.

9. The method of claim 8, wherein the distal end of the biopsy needle extends beyond the distal end of the cannula a minimum of 0.1 cm and a maximum of about 3 cm, thereby preventing the needle from penetrating into the site greater than the distance that the needle extends beyond the trochar.

10. The method of claim 8, wherein the distal end of the biopsy needle is further provided with a cut away portion forming a longitudinally extending cutting edge that is configured to cut tissue in contact with the cutting edge upon rotation of the biopsy needle; the method further comprises inserting the biopsy needle into the site to position the cutting edge against the tissue and rotating the biopsy needle.

11. The method of claim 8, wherein the cannula is an 16 gauge needle or smaller.

12. The method of claim 8, wherein the biopsy needle is a 17 gauge needle or smaller.

13. The method of claim 8, wherein the tissue site is on a lymph node.

14. A method of sampling tissue comprises:
providing a biopsy needle system of claim 2;
inserting a trochar into the cannula;
inserting the cannula and trochar into the patient and positioning the cannula adjacent the site of tissue sampling;
removing the trochar;
inserting the biopsy needle into the cannula;
positioning the tip of the biopsy needle adjacent the site;
inserting the needle into the site of tissue sampling to receive a tissue sample within the second passage; and
removing the needle and the cannula.

15. The method of claim 14 wherein the tissue site is on a lymph node.

16. The method of claim 14, wherein the cannula is a 16 gauge needle or smaller.

17. The method of claim 14, wherein the biopsy needle is a 17 gauge needle or smaller.

18. A kit comprising:
a tubular cannula having a first longitudinal passage, a first length, and a first grip on the proximal end of the cannula, wherein the cannula is sized and configured to receive a biopsy needle;
a tubular biopsy needle having a second longitudinal passage, a second length greater than the first length, a beveled distal end, and a second grip on the proximal end; and
instructions that direct a person to insert the cannula into the patient and position the cannula adjacent to the site of tissue sampling; insert the biopsy needle into the cannula; insert the needle through the cannula into the tissue sample site to receive a tissue sample into the biopsy needle; and remove the needle and cannula.

19. The kit of claim 18, further comprising a trochar that is both sized and configured to fit in the passage of the cannula.
